(19) United States
(12) Patent Application Publication
(10) Pub. No.: US 2007/0078071 A1
(43) Pub. Date: Apr. 5, 2007

(54) Spray dry capsule products and methods for preparing and using same

(76) Inventors: Kaiping Lee, Morganville, NJ (US); Lewis Michael Popplewell, Morganville, NJ (US)

Correspondence Address: INTERNATIONAL FLAVORS & FRAGRANCES INC., 521 WEST 57TH ST, NEW YORK, NY, 10019, US

(21) Appl. No.: 11/240,071

(22) Filed: Sep. 30, 2005

(51) Int. Cl. A61K 8/00

(52) U.S. Cl. 510/130; 510/141

(57) ABSTRACT

The present invention relates to spray-dried compositions comprising encapsulated active materials and a spray-dry carrier, to methods for imparting an effective amount of active materials to spray-dried products and to products incorporating the spray-dried compositions.

### FIELD OF THE INVENTION

The present invention relates to encapsulated compositions in a spray-dried form and to a method for the preparation of these compositions.

### BACKGROUND OF THE INVENTION

Encapsulated fragrances and other active materials are used in consumer products to improve fragrance deposition, retention, and longevity. Often, in order to provide other fragrance benefits to the product or in use, it is required to use a combination of non-confined (free) fragrance and encapsulated fragrance. For example, if a fragrance is well encapsulated, it will provide little contribution to the headspace of a product at point-of-purchase (POP). If POP aroma is important to the consumer purchase decision, then non-confined fragrance may be added to optimize both aroma intensity and hedonics. However, in some applications the base matrix cannot tolerate a substantial amount of added moisture, or base components are prone to plasticization by hydrophobic oils (i.e. fragrance oils). In these cases, the use of encapsulated fragrances and/or benefit agents in an aqueous suspension (slurry form) or fragrance oils alone becomes an issue. Therefore, it is much more desirable to use a dry encapsulated product to deliver fragrances/benefit agents into final consumer products such as powder laundry detergent; fabric softener dryer sheets, household cleaning dry wipes, powder dish detergent, or any dry form of personal care products (e.g. shampoo powder, deodorant powder, foot powder, soap powder, baby powder), etc.

Spray drying is the most commonly used technique for the production of dry powders. While effective in producing traditional encapsulates having a water soluble shell, it does require the use of high process temperatures and exposes dried product to significant shear.

In spray-dried powders, an active ingredient such as a flavor or a fragrance, usually hydrophobic, is entrapped as liquid droplets in a solidified matrix of a dehydrated carrier, generally consisting of carbohydrates, such as starches, hydrolyzed starches (maltodextrin), chemically modified starches, emulsifying polymers (e.g. gum arabic, proteins, etc.) and in certain instances monomers and dimers of simple aldohexoses, or any combination thereof. Conventional spray-drying techniques are perfectly well documented in the prior art. See for example Spray-Drying Handbook, 4.sup.th ed., K. Masters, (1985) or other reference books on the subject-matter.

The method for the preparation of a spray-dried powder typically first comprises the dissolution of a carrier in water, and then the mixture of this solution with a flavor or a fragrance before homogenization to form an oil-in-water emulsion. The emulsion is then spray-dried to produce powdered particles.

Active materials, such as flavors are traditionally spray-dried in water-soluble carbohydrate matrices. As these matrices dissolve in water readily, the most common applications of the corresponding powdered flavors are in dry beverage formulations. However, some applications in the flavor industry as well as in other fields such as the perfumery or the pharmaceutical domains require spray-dried products having a different behavior in an aqueous environment. In particular, powders which would not dissolve as quickly in water and which would even require another trigger mechanism for the control release of the active ingredient encapsulated in the matrix could be very useful for many applications.

Means to improve the dispersion of typical spray-dried powders have already been described in the prior art. Agglomeration constitutes such a means and finds applications for instance in instant coffee. However, this process does not suit all applications.

Surprisingly, it has been found that the spray-dried compositions of the present invention show high retention of the active material, such as a fragrance of about 95% or above, as well as low free fragrance of 0.5% or below if non-confined fragrance is not a part of the formulation. This indicates that the high temperature and shear exposure does not comprise the encapsulation efficiency of the present invention.

### SUMMARY OF THE INVENTION

The present invention provides a composition in spray-dried form comprising a plurality of microcapsules each of which is composed of a polymeric rupturable external wall enclosing an active material dispersed in a spray-dry carrier wherein the carrier comprises a water-soluble material with non-emulsifying and/or self-emulsifying properties, an optional functional additive and an optional non-confined active material with the proviso that when a non-emulsifying water-soluble material is employed in combination with a non-confined active material an emulsifier must be used in the combination; and wherein the composition of each of the cores of each of said microcapsules being (A) the same and/or different from one another and (B) the same and/or different from the non-confined active material optionally present in said spray-dry carrier wherein the weight % of active material initially contained in each of the microcapsules is from about 5% to about 98% by weight of the microcapsules.

In a further embodiment the present invention may also comprise optional functional additives, such as but not limited to, capsule depositions aids, emulsifiers, crosslinkers, humectants, viscosity control agent and free flow agents such as silicas.

In another embodiment of the present invention the encapsulated active material is selected from the group consisting of fragrances, flavoring agents, fungicide, brighteners, antistatic agents, wrinkle control agents, fabric softener actives, hard surface cleaning actives, skin and/or hair conditioning agents, malodour counteractants, antimicrobial actives, UV protection agents, insect repellants, animal/vermin repellents, flame retardants, and mixtures thereof.

In a further embodiment the invention provides a method of imparting an effective amount of an active material into a spray-dried product.

Yet another embodiment provides perfumed, food, beverage and household products comprising the spray-dried composition of the present invention.

### DETAILED DESCRIPTION OF THE INVENTION

The active material suitable for use in the present invention can be a wide variety of materials in which one would want to deliver in a controlled-release manner onto the surfaces being treated with the present compositions or into the environment surrounding the surfaces. Non-limiting examples of active materials include perfumes, flavoring agents, fungicide, brighteners, antistatic agents, wrinkle control agents, fabric softener actives, hard surface cleaning actives, skin and/or hair conditioning agents, antimicrobial actives, UV protection agents, insect repellants, animal/vermin repellants, flame retardants, and the like.

In a preferred embodiment, the active material is a fragrance, in which case the microcapsules containing fragrance provide a controlled-release scent onto the surface being treated or into the environment surrounding the surface. In this case, the fragrance can be comprised of a number of fragrance raw materials known in the art, such as essential oils, botanical extracts, synthetic fragrance materials, and the like.

The present invention is directed to a spray-dried composition comprising of a plurality of microcapsules each of which is composed of a polymeric rupturable external wall enclosing an active material dispersed in a spray-dry carrier wherein the carrier comprises a non-emulsifying water-soluble material such as but not limited to Maltrin M100 (available from Grain Processing Corporation) and/or emulsifying water-souble material such as but not limited to N-LOK (available from National Starch and Chemical Company) and optional functional additives. When in the presence of a non-confined active material a non-emulsifying water-soluble material must be used in combination with an emulsifier such as but not limited to Tween 20. In the presence of an optional non-confined active material, a self-emulsifying water-soluble material may be used alone in the absence of an emulsifier.

The active materials that are not contained in the capsule wall may be added to the capsule slurry before spray drying along with the water soluble matrix materials. This provides a product with two forms of encapsulation. The first (non-confined) active material is dispersed or dissolved in the matrix material, and thus encapsulated. The second (encapsulated) active material is present in the aminoplast capsule, and then further coated by the matrix material. The matrix materials provide a protective layer to improve finished product shelf life, reduce premature release during product storage, and finally to enhance the delivery and release of both the encapsulated and non-confined active materials.

In one embodiment a spray-dried capsule product is provided comprising encapsulated active material or a combination of encapsulated and non-confined active material consisting of:
microcapsules containing encapsulated active material from about 5% to about 98%, more preferably from about 50% to about 97%, most preferably from about 91% to about 96%.
spray dry carrier (i.e. carbohydrates such as chemically modified starches and/or hydrolyzed starches, gums such as gum arabic, proteins such as whey protein, cellulose derivatives, clays, synthetic water-soluble polymers and/or copolymers such as polyvinyl pyrrolidone, polyvinyl alcohol, etc.) from about 1% to about 50%, more preferably from about 5% to about 20%.
Optionally, non-confined active material from about 0.01% to about 50%, more preferably from about 5% to about 45%
Optionally, capsule deposition aid (i.e. cationic starches such as Hi-CAT CWS42, cationic guars such as Jaguar C-162, cationic amino resins, cationic urea resins, hydrophobic quaternary amines, etc.) from about 0.01% to about 25%, more preferably from about 5% to about 20%.
Optionally, emulsifier (i.e. nonionic such as polyoxyethylene sorbitan monostearate (Tween 60), anionic such as sodium oleate, zwitterionic such as lecithins) from about 0.01% to about 25%, more preferably from about 5% to about 10%%.
Optionally, crosslinker (i.e. aminoplasts, aldehydes such as formaldehyde and acetaldehyde, dialdehydes such as glutaraldehyde, epoxy, poly-substituted carboxylic acids and derivatives such as acid chlorides, anyhydrides, isocyanates, tannin/tannic acid, inorganic crosslinkers such as Ca²⁺, etc.) from about 0.01% to about 20%, more preferably from about 0.5% to about 10%.
Optionally, humectant (i.e. polyhydric alcohols such as glycerin, propylene glycol, maltitol, alkoxylated nonionic polymers such as polyethylene glycols, polypropylene glycols, etc.) from about 0.01% to about 25%, more preferably from about 1% to about 5%.
Optionally, viscosity control agent (suspending agent) which may be polymeric or colloidal (i.e. modified cellulose polymers such as methylcellulose, hydoxyethylcellulose, cross-linked acrylate polymers such as Carbomer, hydrophobically modified polyethers, etc.) from about 0.01% to about 25%, more preferably from about 0.5% to about 10%.
Optionally, a free flow agent (anticaking agent) of silicas which may be hydrophobic (i.e. silanol surface treated with halogen silanes, alkoxysilanes, silazanes, siloxanes, etc. such as Sipernat D17, Aerosil R972 and R974 (available from Degussa), etc.) and/or hydrophilic such as Aerosil 200, Sipernat 22S, Sipernat 50S, (available from Degussa), Syloid 244 (available from Grace Davison), etc. from about 0.01% to about 10%, more preferable from 0.5% to about 5%.

Further suitable humectants and viscosity control/suspending agents are disclosed in U.S. Pat. Nos. 4,428,869, 4,464,271, 4,446,032, and 6,930,078. Details of hydrophobic silicas as a functional delivery vehicle of active materials other than a free flow/anticaking agent are disclosed in U.S. Pat. Nos. 5,500,223 and 6,608,017.

The products of the present invention show high retention of the fragrance, as well as low free fragrance indicating that the high temperature and shear exposure does not significantly degrade the encapsulation. Furthermore, with regard to the use of aminoplast chemistry to form the polymeric wall, it has been found that the drying process runs more smoothly if the hygroscopic materials such as urea are not used in the microcapsule slurry formulation as the formaldehyde scavenger. Still further, addition of a catalyst such as magnesium chloride during the capsule polymerization process produces low viscosity slurry that is even more preferred for high efficient/high yield spray drying.

Additionally, as understood by one skilled in the art, a wide range of water-soluble polymers and/or other additives with specific properties can be added to the aminoplast slurry before drying. Use of these matrix materials can further enhance the functionality and stability of encapsulated active materials, modify dry particle size obtained, and improve the delivery and release of the encapsulated fragrances and/or benefit agents.

Composition Variations for Spray Drying

The composition of our invention comprises a (i) a plurality of microcapsules filled with an active material optionally in admixture with a compatible hydrophobic solvent dispersed in (ii) an aqueous emulsion comprising water and a spray dry carrier of non-emulsifying or self-emulsifying materials, and an optional non-confined active material in the presence of an emulsifier. The emulsifier may having a HLB value of from about 6 to about 40 with the provisos that:

(a) when using a non-ionic emulsifier the HLB value is in the range of from about 6 to about 20;

(b) when using an anionic emulsifier, the HLB value is in the range of from about 10 to about 40; and

(c) when using a zwitterionic emulsifier, the HLB value is in the range of from about 6 to about 12.

As disclosed in Application for U.S. Letters patent Ser. No. 10/823,033 filed on Apr. 13, 2004.

For the purpose of creation of the compositions of our invention the emulsifiers, also termed ‘surfactants’ are employed in a concentration of from about 0.1% to about 100% by weight based on the amount of non-confined active material; preferably from about 1% to about 10% by weight based on the amount of non-confined active material, and more preferably at about 2 to about 5% by weight based on the amount of non-confined fragrance active material. As indicated, among the emulsifiers that may be employed are (a) non-ionic emulsifiers having HLB values in the range of from about 6 to about 20, a number of examples of which are set forth in the following Table Ia together with their respective HLB values:

TABLE Ia

| Common Name(“TWEEN”, | | |
|---|---|---|
| “SPAN” and “ATLAS” | | |
| are the registered trademarks | | |
| of ICI Americas Inc. of | | |
| Bridgewater, N.J.) | Chemical Designation | HLB Value |
| SPAN 40 | Sorbitan monpalmitate | 6.7 |
| ATLAS G-2800 | Polyoxypropylene | 8.0 |
| | mannitol dioleate | |
| PEG 400 monolaurate | polyoxyethylene | 13.1 |
| | monolaurate | |
| TWEEN 60 | polyoxyethylene sorbitan | 14.9 |
| | monostearate | |
| TWEEN 40 | polyoxyethylene sorbitan | 15.6 |
| | monopalmitate | |
| TWEEN 20 | polyoxyethylene sorbitan | 16.7 |
| | monolaurate | |
| ATLAS G-2159 | polyoxyethylene | 18.8 |
| | monostearate | |

(b) anionic emulsifiers having HLB values in the range of from about 10 to about 40, a number of examples of which are set forth in the following Table Ib together with their respective HLB values:

TABLE Ib

| Common Name | Chemical Name | HLB Value |
|---|---|---|
| ATLAS G-3300 | An alkyl aryl sulfonate | 11.7 |
| Triethanolamine oleate | Triethanolamine oleate | 12 |
| Sodium Oleate | Sodium Oleate | 18 |
| Potassium Oleate | Potassium Oleate | 20 |
| Sodium Lauryl Sulfate | Sodium Lauryl Sulfate | 40 |

(c) zwitterionic emulsifiers having HLB values in the range of from about 6 to about 12, which are lecithins containing one or more phosphatidyl cholines, phosphatadylethanolamines and/or phosphatidylinositols, a number of examples of which are set forth in the following Table Ic, together with their respective HLB values:

TABLE Ic

| Common Name (All Registered Trademarks of Central | |
|---|---|
| Soya Company Inc. of Fort Wayne, Indiana) | HLB Value |
| Centrophase HR 4B | 7.5 |
| Blendmax K | 8.0 |
| Centrolene A | 10 |
| Centromix E | 12 |
| Centromix CPS | 12 |

(d) polymeric emulsifiers, a number of examples of which are set forth in the following Table Id:

TABLE Id

Name
Gum Arabic, Colloides Naturale
Capsul Modified Starch, National Starch & Chemical
Pemulen, Noveon Inc., a high molecular weight, cross linked
copolymers of acrylic acid and a hydrophobic comonomer

Additionally, water-soluble spray dry carriers with non-emulsifying properties can be selected from polymers including carbohydrates such as, but not limited to, methylcellulose, hydroxyethylcellulose, hydroxypropylcellulose, gums such as, but not limited to, agar, xanthan, sodium alginate, proteins such as, but not limited to, gelatin, synthetic polymers and/or copolymers such as, but not limited to, polyvinyl pyrrolidone, polyvinyl alcohol, ethylene maleic anhydride copolymer, and from mono, di and trisaccharides such as, but not limited to glucose, fructose, maltose, lactose, sucrose, dextrinized and hydrolyzed starches such as, but not limited to maltodextrins, corn syrup solids, glycols such as, but not limited to, polyethylene glycol and combinations thereof.

Furthermore, water-soluble spray dry carriers with self-emulsifying properties can be selected from polymers, in accordance with the present invention, including carbohydrates such as, but not limited to, modified starches, gums such as, but not limited to, gum arabic, proteins such as, but not limited to, soy protein, whey protein, modified wheat protein, and combinations thereof.

Further suitable water soluble and water dispersible polymers as a spray dry carrier are disclosed in U.S. Pat. No 6,740,631.

Microcapsules

Encapsulation of active materials such as fragrances is known in the art, see for example U.S. Pat. Nos. 2,800,457, 3,870,542, 3,516,941, 3,415,758, 3,041,288, 5,112,688, 6,329,057, and 6,261,483. Another discussion of fragrance encapsulation is found in the Kirk-Othmer Encyclopedia.

Preferred encapsulating polymers include those formed from melamine-formaldehyde or urea-formaldehyde condensates, as well as similar types of aminoplasts. Additionally, capsules made via the simple or complex coacervation of gelatin are also preferred for use with the coating. Capsules having shell walls comprised of polyurethane, polyamide, polyolefin, polysaccaharide, protein, silicone, lipid, modified cellulose, gums, polyacrylate, polystyrene, and polyesters or combinations of these materials are also functional.

A representative process used for aminoplast encapsulation is disclosed in U.S. Pat. No. 3,516,941 though it is recognized that many variations with regard to materials and process steps are possible. A representative process used for gelatin encapsulation is disclosed in U.S. Pat. No. 2,800,457 though it is recognized that many variations with regard to materials and process steps are possible. Both of these processes are discussed in the context of fragrance encapsulation for use in consumer products in U.S. Pat. Nos. 4,145,184 and 5,112,688 respectively.

Well known materials such as solvents, surfactants, emulsifiers, and the like can be used in addition to the polymers described above to encapsulate the active materials such as fragrance without departing from the scope of the present invention. It is understood that the term encapsulated is meant to mean that the fragrance material is substantially covered in its entirety. Encapsulation can provide pore vacancies or interstitial openings depending on the encapsulation techniques employed. More preferably the entire fragrance material portion of the present invention is encapsulated.

Fragrance capsules known in the art consists of a core of various ratios of fragrance and a diluent, a wall or shell comprising a three-dimensional cross-linked network of an aminoplast resin, more specifically a substituted or un-substituted acrylic acid polymer or co-polymer cross-linked with a urea-formaldehyde pre-condensate or a melamine-formaldehyde pre-condensate.

The microcapsule walls are preferably composed of an aminoplast resin, more specifically a substituted or un-substituted acrylic acid polymer or co-polymer cross-linked with a urea-formaldehyde pre-condensate or a melamine-formaldehyde pre-condensate. The microcapsule is formed by means of either (a) forming an aqueous dispersion of a non-cured aminoplast resin by reacting under acidic pH conditions a urea-formaldehyde pre-condensate or a melamine-formaldehyde pre-condensate with one or more substituted or un-substituted acrylic acid polymers or co-polymers; then coacervating the resulting non-cured aminoplast resin shell around the surface of a fragrance and/or malodour counteractant-solvent monophasic droplet under homogenization conditions, e.g., using a homogenization apparatus as described in U.S. Pat. No. 6,042,792 and illustrated in FIGS. 7A to 7D and 8A to 8E of U.S. patent application No. 10/823,033, and then curing the microcapsule shell wall at an elevated temperature, e.g., 50-85° C. or (b) forming the aminoplast resin wall at the surface of the fragrance and/or malodour counteractant-solvent monophasic droplet by means of reacting, at the surface of the droplet a urea-formaldehyde pre-condensate or a melamine-formaldehyde pre-condensate with one or more substituted or un-substituted acrylic acid polymers or co-polymers, and then curing the microcapsule shell wall at an elevated temperature, e.g., 50 - 85° C.

Microcapsule formation using mechanisms similar to the foregoing mechanism, using (i) melamine-formaldehyde or urea-formaldehyde pre-condensates and (ii) polymers containing substituted vinyl monomeric units having proton-donating functional group moieties, e.g., sulfonic acid groups or carboxylic acid anhydride groups, bonded thereto is disclosed in U.S. Pat. No. 4,406,816 (2-acrylamido-2-methyl-propane sulfonic acid groups), UK published Patent Application GB 2,062,570 A (styrene sulfonic acid groups) and UK published Patent Application GB 2,006,709 A (carboxylic acid anhydride groups).

When substituted or un-substituted acrylic acid co-polymers are employed in the practice of our invention, in the case of using a co-polymer having two different monomeric units, e.g., acrylamide monomeric units and acrylic acid monomeric units, the mole ratio of the first monomeric unit to the second monomeric unit is in the range of from about 1:9 to about 9:1, preferably from about 3:7 to about 7:3. In the case of using a co-polymer having three different monomeric units, e.g., ethyl methacrylate, acrylic acid and acrylamide, the mole ratio of the first monomeric unit to the second monomeric unit to the third monomeric unit is in the range of 1:1:8 to about 8:8:1, preferably from about 3:3:7 to about 7:7:3.

As disclosed in copending Application for U.S. Letters patent Ser. No. 10/823,492 filed on Apr. 13, 2004.

The molecular weight range of the substituted or un-substituted acrylic acid polymers or co-polymers useful in the practice of our invention is from about 5,000 to about 1,000,000, preferably from about 10,000 to about 100,000. The substituted or un-substituted acrylic acid polymers or co-polymers useful in the practice of our invention may be branched, linear, star-shaped, dendritic-shaped or may be a block polymer or copolymer, or blends of any of the aforementioned polymers or copolymers.

Such substituted or un-substituted acrylic acid polymers or co-polymers may be prepared according to any processes known to those skilled in the art, for example, U.S. Pat. No. 6,545,084.

The urea-formaldehyde and melamine-formaldehyde pre-condensate microcapsule shell wall precursors are prepared by means of reacting urea or melamine with formaldehyde where the mole ratio of melamine or urea to formaldehyde is in the range of from about 10:1 to about 1:6, preferably from about 1:2 to about 1:5. For purposes of practicing our invention, the resulting material has a molecular weight in the range of from 156 to 3000. The resulting material may be used as a cross-linking agent for the aforementioned substituted or un-substituted acrylic acid polymer or copolymer or it may be further reacted with a C₁-C₆ alkanol, e.g., methanol, ethanol, 2-propanol, 3-propanol, 1-butanol , 1-pentanol or 1-hexanol, thereby forming a partial ether where the mole ratio of melamine or urea:formalhyde:alkanol is in the range of 1:(0.1-6):(0.1-6). The resulting ether moiety-containing product may by used as a cross-linking agent for the aforementioned substituted or un-substituted acrylic acid polymer or copolymer, or it may be self-condensed to form dimers, trimers and/or tetramers which may also be used as cross-linking agents for the aforementioned substituted or un-substituted acrylic acid polymers or co-polymers. Methods for formation of such melamine-formaldehyde and urea-formaldehyde pre-condensates are set forth in U.S. Pat. No. 3,516,846, U.S. Pat. No. 6,261,483, and Lee et al. J. Microencapsulation, 2002, Vol. 19, No. 5, pp 559-569, “Microencapsulation of fragrant oil via in situ polymerization: effects of pH and melamine-formaldehyde molar ratio”. Examples of urea-formaldehyde pre-condensates useful in the practice of our invention are URAC 180 and URAC 186, Cytec Technology Corp. Examples of melamine-formaldehyde pre-condensates useful in the practice of our invention are CYMEL U-60, CYMEL U-64 and CYMEL U-65, Cytec Technology Corp.

In practicing our invention, the range of mole ratios of urea-formaldehyde precondensate or melamine-formaldehyde pre-condensate:substituted or un-substituted acrylic acid polymer or co-polymer is in the range of from about 9:1 to about 1:9, preferably from about 5:1 to about 1:5 and most preferably from about 1:2 to about 2:1.

In another embodiment of the invention, capsules with polymer(s) comprising primary and/or secondary amine reactive groups or mixtures thereof and crosslinkers are provided. The amine polymers can possess primary and/or secondary amine functionalities and can be of either natural or synthetic origin. Amine containing polymers of natural origin are typically proteins such as gelatin and albumen, as well as some polysaccharides. Synthetic amine polymers include various degrees of hydrolyzed polyvinyl formamides, polyvinylamines, polyallyl amines and other synthetic polymers with primary and secondary amine pendants. Examples of suitable amine polymers are the Lupamin series of polyvinyl formamides (available from BASF). The molecular weights of these materials can range from 10,000 to 1,000,000.

As disclosed in copending Application for U.S. Letters patent Ser. No. 11/123,898 filed on May 6, 2005.

The polymers containing primary and/or secondary amines can be used with any of the following comonomers in any combination:

1. Vinyl and acrylic monomers with:
a. alkyl, aryl and silyl substituents;
b. OH, COOH, SH, aldehyde, trimonium, sulfonate, NH₂, NHR substiuents;
c. vinyl pyridine, vinyl pyridine-N-oxide, vinyl pyrrolidon

2. Cationic monomers such as dialkyl dimethylammonium chloride, vinyl imidazolinium halides, methylated vinyl pyridine, cationic acrylamides and guanidine-based monomers

3. N-vinyl formamide

and any mixtures thereof. The ratio amine monomer/total monomer ranges from 0.01-0.99, more preferred from 0.1-0.9.

The following represents a general formula for the amine-containing polymer material:

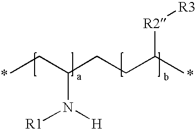

wherein R is a saturated or unsaturated alkane, dialkylsiloxy, dialkyloxy, aryl, alkylated aryl, and that may further contain a cyano, OH, COOH, NH₂, NHR, sulfonate, sulphate, —NH₂, quaternized amines, thiols, aldehyde, alkoxy, pyrrolidone, pyridine, imidazol, imidazolinium halide, guanidine, phosphate, monosaccharide, oligo or polysaccharide.

R1 is H, CH₃, (C═O)H, alkylene, alkylene with unsaturated C—C bonds, CH₂—CROH, (C═O)—NH—R, (C═O)—(CH₂)n-OH, (C═O)—R, (CH₂)n-E, —(CH₂-CH(C═O))n-XR, —(CH₂)n-COOH, —(CH₂)n-NH₂, —CH₂)n-(C═O)NH₂, E is an electrophilic group; wherein a and b are integers or average numbers (real numbers) from about 100-25,000.

R2 can be nonexistent or the functional group selected from the group consisting of —COO—, —(C═O)—, —O—, —S—, —NH—(C═O)—, —NR1—, dialkylsiloxy, dialkyloxy, phenylene, naphthalene, alkyleneoxy. R3 can be the same or selected from the same group as R1.

Additional copolymers with amine monomers are provided having the structure: 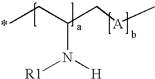
R1 is H, CH₃, (C═O)H, alkylene, alkylene with unsaturated C—C bonds, CH₂—CROH, (C═O)—NH—R, (C═O)—(CH₂)n-OH, (C═O)—R, (CH₂)n-E, —(CH₂—CH(C═O))n-XR, —(CH₂)n-COOH, —(CH₂)n-NH₂, —CH₂)n-(C═O)NH₂, E is an electrophilic group; wherein a and b are integers or average numbers (real numbers) from about 100-25,000.

The comonomer, represented by A, can contain an amine monomer and a cyclic monomer wherein A can be selected from the group consisting of aminals, hydrolyzed or non-hydrolyzed maleic anhydride, vinyl pyrrolidine, vinyl pyridine, vinyl pyridine-N-oxide, methylated vinyl pyridine, vinyl naphthalene, vinyl naphthalene-sulfonate and mixtures thereof.

When A is an aminal the following general structure can represent the aminal:

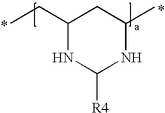

wherein R4 is selected from the group consisting of H, CH₃, (C═O)H, alkylene, alkylene with unsaturated C—C bonds, CH₂—CROH, (C═O)—NH—R, (C═O)—(CH₂)n-OH, (C═O)-R, (CH₂)n-E, —(CH₂—CH(C═O))n-XR, —(CH₂)n-COOH, —(CH₂)n-NH2, —CH₂)n-(C═O)NH₂, E is an electrophilic group; wherein R is a saturated or unsaturated alkane, dialkylsiloxy, dialkyloxy, aryl, alkylated aryl, and that may further contain a cyano, OH, COOH, NH₂, NHR, sulfonate, sulphate, —NH₂, quaternized amines, thiols, aldehyde, alkoxy, pyrrolidone, pyridine, imidazol, imidazolinium halide, guanidine, phosphate, monosaccharide, oligo or polysaccharide.

In addition instead of amine-containing polymers it is possible to utilize amine-generating polymers that can generate primary and secondary amines during the capsule formation process.

The benefits of the preferred embodiment are that these capsules have better encapsulated fragrance and/or benefit agent leaching stability in certain product bases as compared to standard aminoplast capsules. Additional benefits are that the capsules can deposit better on their own as because (i) they have the potential of being cationically charged at pH of about 8 and less, and/or (ii) the improved adhesion force of unreacted amine functionalities. The capsules also have less interaction with anionic surfactant as the capsule surface is not strongly positively charged. These additional benefits eliminate the need for deposition aids in specific applications.

The crosslinkers can be selected from the group consisting of aminoplasts, aldehydes such as formaldehyde and acetaldehyde, dialdehydes such as glutaraldehyde, epoxy, active oxygen such as ozone and OH radicals, poly-substituted carboxylic acids and derivatives such as acid chlorides, anyhydrides, isocyanates, diketones, halide-substituted, sulfonyl chloride-based organics, inorganic crosslinkers such as Ca²⁺, organics capable of forming azo, azoxy and hydrazo bonds, lactones and lactams, thionyl chloride, phosgene, tannin/tannic acid, polyphenols and mixtures thereof. Furthermore, processes such as free radical and radiation crosslinking can be used according to the present invention. Examples of free radical crosslinkers are benzoyl peroxide, sodium persulfate, azoisobutylnitrile (AIBN) and mixtures thereof.

In a further embodiment a crosslinker can be added to the encapsulated material once the reaction has completed. The additional crosslinker reacts with itself and also with the unreacted groups on the capsule surface.

The encapsulated active material and/or odor controlling agents with amine-containing polymers provides the advantage that the encapsulated materials contain a core and a shell and do not require further coating by a cationic polymer thereby saving processing time and the additional costs incurred in processing.

The microcapsule walls can be composed of an amine-containing polymer and/or amine-generating polymer which may be combined with a comonomers and mixtures thereof, cross-linked with a crosslinker such as but not limited to formaldehyde pre-condensate such as a urea or melamine-formaldehyde pre-condensate.

The average outside diameter of the resulting microcapsule is in the range of from about 0.01 microns to about 1000 microns; preferably from about 0.05 microns to about 100 microns and more preferably from about 2.0 microns to about 20 microns. The average wall thickness of the resulting microcapsule is in the range of from about 0.001 microns to about 100 microns; preferably from about 0.005 microns to about 10 microns and more preferably from about 0.2 microns to about 2.0 microns.

Optionally, in order to provide an increased period of time during which the microencapsulates are retained on surfaces to be treated using the consumable products into which the dry product of our invention are incorporated, the aminoplast microencapsulates used in the practice of our invention may be coated with a cationic polymer as disclosed in Application for U.S. Letters Patent Publication Number 2005/0112152 filed on Nov. 20, 2003 and, in addition, Applications for U.S. Patent Publication Numbers 2004/0071742 and 2004/0072719 filed on Oct. 10, 2002. The rate of use of such cationic polymer coatings on the microencapsulates is from about 1% to about 3000% by weight of the filled microencapsulates; preferably from about 5% to about 1000% by weight of the filled microencapsulates; and most preferably from about 10% to about 500% by weight of the filled microencapsulates.

Examples of such cationic polymers used as coatings are cationically modified starch and cationically modified guar, polymers comprising poly diallyl dimethyl ammonium halides (PolyDADMAC), and copolymers of DADMAC with vinyl pyrrolidone, acrylamides, imidazoles, imidazolinium halides, and the like. For instance, Polyquaternium-6, 7, 22 and 39, all available from Ondeo Nalco.

The preferred cationic starch has a molecular weight of from about 100,000 to about 500,000,000, preferably from about 200,000 to about 10,000,000 and most preferably from about 250,000 to about 5,000,000. The preferred cationic starch products are HI-CAT CWS42 and HI-CAT 02 and are commercially available from ROQUETTE AMERICA, Inc.

The preferred cationic guar has a molecular weight of from about 50,000 to about 5,000,000. The preferred cationic guar products are Jaguar C-162 and Jaguar C-17 and are commercially available from Rhodia Inc.

Additional examples of cationic polymers useful for coating the aminoplast encapsulated solvent/fragrance compositions and/or solvent/malodour counteractant compositions of our invention are the water-soluble cationic amino resins, cationic urea resins, specifically, urea-formaldehyde pre-polymers subjected to polycondensation with a cationic modifier such as diethylenetriamine, tetraethylene pentamine, guanidine, guanyl urea and oxazolidine as disclosed in Application for U.S. Letters Patent Publication Number 2001/008874 filed on Apr. 13, 2004 and published on Jul. 19, 2001, for example, U-RAMIN P-1500, Mitsui Kagaku K.K., a urea-formaldehyde pre-polymer modified with diethylene triamine.

According to the present invention the capsule process can be catalyzed by acids and/or metal salts and mixtures thereof, to produce better performance and stability in a base, such as, but not limited to, a fabric conditioner, dry and liquid detergent, tumbler dryer sheets, shampoo, body lotion, body wash, hard surface cleaners, soap bars, hair conditioners, hair fixatives, hair color and dyes and after-shave lotions. In additional these capsules may be applied (physical or chemically) on textile fabrics during manufacture.

The role of the acid catalyst is two-fold: first it causes the prepolymer to build viscosity, which is necessary for capsule formation, and second, it catalyzes the curing crosslinking reaction. The standard acid catalyst used in the current process known in the art is acetic acid at a level of 6 to 7% trs (total resin solid), which results in a pH of 5.0.

As discussed above, the rate of the viscosity buildup of the prepolymer is a function of pH, with the rate increasing as a function of decreasing pH. The pH may be adjusted using the acid catalyst. The catalysts may be both weak and strong organic and mineral acids.

Examples of acid catalyst include, but are not limited to, hydrochloric (HCl), sulfuric, phosphoric, para-toluenesulfonic (pTSA), acetic, glycolic, lactic, benzoic, citric, maleic, and commercially available catalysts from the coatings and textile industries (Nacure XP-333 (available from King Industries), K-Cure 129W (available from King Industries), Cycat 296-9 (available from Cytec), Polystep A-13 (available from Stepan).

In addition to affecting the prepolymer viscosity buildup, these acids also affect the capsule performance by participating in the crosslinking reaction, acting as crosslinkers themselves. The aforementioned acids can be grouped according to their functionalities: monoprotic, diprotic, and triprotic. Examples of suitable diprotic acids are oxalic and maleic. A trifunctional acid includes citric acid.

Capsule performance is sensitive to the degree of crosslinking that the multifunctional acids impart. Therefore it may be necessary to adjust these acid levels up or down to optimize performance.

In addition to acids, metal salts can also be used to enhance capsule stability and performance, alone or in combination with the acid catalysts. By themselves the metal salts act as Lewis acids which enhance the crosslinking. In combination with alpha-hydroxy acids, such as, citric, glycolic, lactic, there is an enhanced catalytic effect due to the metal salt's coordination with the hydroxyl group on the acid. These salts can be employed in levels that range from about 1 to about 15% trs.

The metal salts can be selected from the group consisting of but not limited to ammonium chloride (NH₄Cl), aluminum nitrate (Al(NO₃)₃), aluminum sulfate (Al(SO₄)₃), magnesium bromide (MgBr₂), magnesium chloride (MgCl₂), magnesium nitrate (Mg(NO₃)₂), zinc bromide (ZnBr₂), zinc iodide (ZnI₂), and zirconyl nitrate (ZrO(NO₃)₂) . Preferred metal salts include NH₄Cl, MgBr₂, MgCl₂, Mg(NO₃)₂, and ZnI₂. More preferred metal salts include MgBr₂, MgCl₂, Mg(NO₃)₂, and ZnI₂ result in performance gains.

In a further embodiment, alpha-hydroxyacids such as lactic, glycolic, and citric lactic performance have a synergistic effect with metal salts such as, but not limited to MgBr₂, MgCl₂, Mg(NO₃)₂, ZnI₂ and mixtures thereof.

Active Materials in Microcapsules

The active material of the resulting microcapsule includes a fragrance composition and/or a benefit agent such as a malodour counteractant composition in combination with a compatible hydrophobic solvent. The term “compatible” is herein intended to mean essentially chemically non-reactive with every fragrance component and/or benefit agent such as a malodour counteractant component and preferably capable of forming a single liquid phase with each fragrance composition component and with each benefit agent component such as a malodour counteractant composition component.

In the practice of our invention, the range of weight percent of solvent/fragrance composition components and/or solvent/malodour counteractant composition components contained in each of the microcapsules is from about 5% to about 98%, preferably from about 50 to about 97% by weight of the microcapsule, most preferably from about 91% to about 96%. Thus, the range of weight ratios of encapsulating polymer to solvent/fragrance composition components and/or solvent/malodour counteractant components is preferably from about 1:25 to about 1:1; most preferably from about 1:10 to about 4:96. In addition, the range of weight percent of solvent in the microcapsule is preferably from about 10% to 80% by weight of the filled microcapsule. In a highly preferred ratio of weight of solvent:weight of encapsulated fragrance composition and/or encapsulated malodour counteractant composition is from about 2:1 to about 1:2, with the most preferred ratio being 1:1.

The compatible hydrophobic solvent used in combination with the microencapsulated fragrance composition and/or microencapsulated benefit agent, e.g., malodour counteractant composition is preferably a mono-, di- or tri-C₄-C₂₆ saturated or unsaturated fatty acid glyceride, diethyl phthalate, dibutyl phthalate, diisodecyl adipate, a liquid polydimethyl siloxane, a liquid polydimethylcyclosiloxane, the methyl ester of soya fatty acid, a mixture of soya fatty acid methyl ester and isopropyl myristate with the weight ratio of soya fatty acid:isopropyl myristate being from 2:1 to 20:1 and a mineral oil compatible with each component of said fragrance composition and/or said benefit agent, e.g., malodour counteractant composition. More preferably, the solvent is a tri-C₄-C₂₆ saturated or unsaturated fatty acid glyceride. Most preferably, the solvent is the tri-glyceride ester of a mixture of caprylic acid and capric acid, commercially available as NEOBEE M-5, Stepan Chemical Company. The C log₁₀P of the solvent is greater than 3.3, where P is the n-octanol/water partition coefficient of the hydrophobic solvent; preferably greater than about 8 and most preferably greater than about 10.

The Clog₁₀P of each component of the encapsulated fragrance composition and/or the encapsulated malodour counteractant composition preferably is in the range of from about 3.3 to about 8, where P is the n-octanol/water partition coefficient of the fragrance component, although relatively low percentages of fragrance components having a lower value of C log₁₀P may be used in conjunction with the components having a C log₁₀P of between 3.3 and 8. In a preferred embodiment the fragrance or benefit agent is free of a solid material, but does not preclude the inclusion of crystals, particles and the like.

The performance of the capsules of the present invention may be improved through the use of a vast preponderance of high ClogP fragrance materials. In this embodiment of the invention greater than about 60 weight percent of the encapsulated fragrance materials have a ClogP of greater than 3.3. In another highly preferred embodiment of the invention more than 80 weight percent of the encapsulated fragrances have a ClogP value of greater than about 4.0. Use of fragrance materials as described previously reduces the diffusion of fragrance through the capsule wall and into the consumer product base under specific time, temperature, and concentration conditions.

The higher ClogP materials are preferred, meaning that those materials with a ClogP value of 4.5 are preferred over those fragrance materials with a ClogP of 4; and those materials are preferred over the fragrance materials with a ClogP of 3.3.

The encapsulated fragrance formulation of the present invention should have at least about 60 weight percent of materials with ClogP greater than 3.3, preferably greater than about 80 and more preferably greater than about 90 weight percent of materials with ClogP greater than 4.

As disclosed in Application for U.S. Letters Patent Publication Number 2005/0112152 filed on Nov. 20, 2003 and published on May 20, 2005.

Those with skill in the art appreciate that fragrance formulations are frequently complex mixtures of many fragrance ingredients. A perfumer commonly has several thousand fragrance chemicals to work from. Those with skill in the art appreciate that the present invention may contain a single ingredient, but it is much more likely that the present invention will comprise at least eight or more fragrance chemicals, more likely to contain twelve or more and often twenty or more fragrance chemicals. The present invention also contemplates the use of complex fragrance formulations containing fifty or more fragrance chemicals, seventy five or more or even a hundred or more fragrance chemicals in a fragrance formulation.

Preferred fragrance materials will have both high ClogP and high vapor pressure.

Para cymene, Caphene, Mandarinal Firm, Vivaldie, Terpinene, Verdox, Fenchyl acetate, Cyclohexyl isovalerate, Manzanate, Myrcene, Herbavert, Isobutyl isobutyrate, Tetrahydrocitral, Ocimene and Caryophyllene.

The values of C log₁₀P of many functional product ingredients, for example, fragrance ingredients contained in personal treatment compositions and/or cosmetic compositions is discussed in U.S. Pat. Nos. 5,783,544, 6,528,013, 6,656,923 and 6,652,766. Furthermore, values of log₁₀P have been reported; for example, the Pomona92 database, available from Daylight Chemical Information Systems, Inc., Daylight CIS, Irvine, Calif. However, the log₁₀P values are most conveniently calculated by the “CLOGP” program, also available from Daylight CIS. This program also lists experimental log₁₀P values when they are available in the Pomona92 database. The “calculated log₁₀P” (C log₁₀P) is determined by the Hansch and Leo “fragment” approach based on the chemical structure of each functional product ingredient, and takes into account the numbers and types of atoms, the atom connectivity and the chemical bonding. The C log₁₀P values which are the most reliable and widely used estimates for this physicochemical property, are preferably.used instead of the experimental log₁₀P values for the selection of functional ingredients, including perfume ingredients which are useful components in the microencapsulate-containing slurries of our invention.

Specific examples of preferred fragrance components useful in the aminoplast microencapsulates used in the composition and process of our invention, and the molecular weights and Clog₁₀P values of each of said components are set forth in Table II as follows:

TABLE II

| | Clog₁₀P | Molecular |
|---|---|---|
| Fragance Component | value | Weight |
| amyl salicylate | 4.601 | 208.26 |
| benzyl salicylate | 4.383 | 228.25 |
| β-caryophyllene | 6.333 | 204.36 |
| ethyl undecylenate | 4.888 | 212.34 |
| geranyl anthranilate | 4.216 | 273.38 |
| α-irone | 3.820 | 206.33 |
| β-phenyl ethyl benzoate | 4.058 | 226.28 |
| α-santalol | 3.800 | 220.36 |
| amyl salicylate | 4.601 | 208.26 |
| β-caryophyllene | 6.333 | 204.36 |
| cedrol | 4.530 | 222.37 |
| cedryl acetate | 5.436 | 264.41 |
| cedryl formate | 5.070 | 238.37 |
| cyclohexyl salicylate | 5.265 | 220.29 |
| γ-dodecalactone | 4.359 | 198.31 |
| β-phenylethyl phenyl acetate | 3.767 | 240.31 |
| 5-acetyl-1,1,2,3,3,6-hexamethyl indane | 5.977 | 258.41 |
| cyclopentadecanolide | 6.246 | 240.39 |
| amyl cinnamic aldehyde | 4.324 | 202.30 |
| linalyl benzoate | 5.233 | 258.36 |

Specific examples of malodour counteractant composition components useful in the aminoplast microencapsulates used in the composition and process of our invention are as follows:

Malodour Counteractant Component Group I:

1-cyclohexylethan-1-yl butyrate;

1-cyclohexylethan-1-yl acetate;

1-cyclohexylethan-1-ol;

1-(4′-methylethyl)cyclohexylethan-1-yl propionate; and

2′-hydroxy-1′-ethyl(2-phenoxy)acetate

each of which compound is marketed under the trademark VEILEX® by International Flavors & Fragrances Inc., New York, N.Y. Malodour Counteractant Component Group II, as disclosed in U.S. Pat. No. 6,379,658:

β-naphthyl methyl ether;

β-naphthyl ketone;

benzyl acetone;

mixture of hexahydro-4,7-methanoinden-5-yl propionate and hexahydro-4,7-methanoinden-6-yl propionate;

4-(2,6,6-trimethyl-2-cyclohexen-1-yl)-3-methyl-3-buten-2-one;
3,7-dimethyl-2,6-nonadien-1-nitrile;
dodecahydro-3a,6,6,9a-tetramethylnaphtho(2,1-b)furan;
ethylene glycol cyclic ester of n-dodecanedioic acid;
1-cyclohexadecen-6-one;
1-cycloheptadecen-10-one; and
corn mint oil.

Insect repellent agents useful in the practice of our invention are disclosed in Published Application for U.S. Patent 2003/0005522 A1 published on Jan. 9, 2003. Preferred insect repellent components useful in the practice of our invention are geraniol, geranium oil, citral and nerol.

Active material as defined by the present invention embraces fragrances, flavoring agents, fungicide, brighteners, antistatic agents, wrinkle control agents, fabric softener actives, hard surface cleaning actives, skin and/or hair conditioning agents, malodour counteractant, antimicrobial actives, UV protection agents, insect repellants, animal/vermin repellants, flame retardants, and mixtures thereof.

Non-Confined Active Material and Functional Additive

The non-confined active material such as, but not limited to, fragrance and/or benefit agent composition in the stable suspension of our invention is contained in the “oil-in-water” emulsion in which the microencapsulated fragrance and/or benefit agent is suspended. The C log₁₀P range of each of the non-confined fragrance and/or benefit agent components is in the range of from about 1 to about 15 thus enabling a greater range of fragrance and/or benefit agent component types in the non-confined fragrance and/or benefit agent as opposed to the components of the ‘confined’ or ‘microencapsulated’ fragrance and/or benefit agent.

Within the scope of our invention, each of the oil phase component droplets of the emulsion containing non-confined fragrance and/or benefit agent has a diameter in the range of from about 0.01 to about 10 microns; preferably in the range of from about 0.05 to about 0.8 microns, and more preferably in the range of from about 0.1 to about 0.5 microns.

Specific examples of non-confined fragrance components, their molecular weights and their C log₁₀P's are set forth in the following Table III:

TABLE III

| | Clog₁₀P | Molecular |
|---|---|---|
| Fragance Component | value | Weight |
| benzaldehyde | 1.480 | 106.12 |
| benzyl acetate | 1.960 | 150.17 |
| laevo-carvone | 2.083 | 150.22 |
| geraniol | 2.649 | 154.26 |
| cis-jasmone | 2.712 | 164.25 |
| β-phenylethyl alcohol | 1.183 | 122.17 |
| α-terpineol | 2.569 | 154.25 |
| 1-phenyl hexanol-5 | 3.299 | 178.28 |
| dihydromyrcenol | 3.03 | 156.27 |
| δ-undecalactone | 3.830 | 184.28 |
| amyl cinnamate | 3.771 | 218.30 |
| benzophenone | 3.120 | 182.22 |
| nerol | 2.649 | 154.25 |
| 2-methoxynaphthalene | 3.235 | 158.20 |
| ethyl undecylenate | 4.888 | 212.34 |
| geranyl anthranilate | 4.216 | 273.38 |
| α-irone | 3.820 | 206.33 |
| α-santalol | 3.800 | 220.36 |
| iso-eugenol | 2.547 | 164.21 |
| amyl salicylate | 4.601 | 208.26 |
| benzyl salicylate | 4.383 | 228.25 |
| β-caryophyllene | 6.333 | 204.36 |
| cedrol | 4.530 | 222.37 |
| cedryl acetate | 5.436 | 264.41 |
| cedryl formate | 5.070 | 238.37 |
| cyclohexyl salicylate | 5.265 | 220.29 |
| γ-dodecalactone | 4.359 | 198.31 |
| ethyl undecylenate | 4.888 | 212.34 |
| geranyl anthranilate | 4.216 | 273.38 |
| β-phenylethyl benzoate | 4.058 | 226.38 |
| β-phenylethyl phenyl acetate | 3.767 | 240.31 |
| 5-acetyl-1,1,2,3,3,6-hexamethyl indane | 5.977 | 258.41 |
| cyclopentadecanolide | 6.246 | 240.39 |
| d-limonene | 4.232 | 136.24 |
| cis-p-t-butylcyclohexyl acetate | 4.019 | 198.31 |
| amyl cinnamic aldehyde | 4.324 | 202.30 |

The suspension containing the confined and non-confined fragrance and/or benefit agent may also contain at least one of the following auxiliary substances in amounts of from about 0.01% to about 30% by weight of the non-confined fragrance and/or benefit agent composition:

Optionally, at least one capsule deposition aid (i.e. cationic starches such as Hi-CAT CWS42, cationic guars such as Jaguar C-162, cationic amino resins, cationic urea resins, hydrophobic quaternary amines, etc.);

Optionally, at least one additional emulsifier (i.e. nonionic such as polyoxyethylene sorbitan monostearate (Tween 60), anionic such as sodium oleate, zwitterionic such as lecithins);

Optionally, at least one additional crosslinker (i.e. aminoplasts, aldehydes such as acetaldehyde, dialdehydes such as glutaraldehyde, poly-substituted carboxylic acids and derivatives such as acid chlorides, anyhydrides, isocyanates, tannin/tannic acid, etc.);

Optionally, at least one humectant (i.e. polyhydric alcohols such as glycerin, propylene glycol, maltitol, alkoxylated nonionic polymers such as polyethylene glycols, polypropylene glycols, etc.);

Optionally, at least one viscosity control agent (i.e. modified cellulose polymers such as methylcellulose, hydoxyethylcellulose, cross-linked acrylate polymers such as Carbomer, hydrophobically modified polyethers, etc.);

Optionally, at least one hydrophobic solvent (i.e. mono-, di- or tri-C₄-C₂₆ saturated or unsaturated fatty acid glyceride, diethyl phthalate, dibutyl phthalate, diisodecyl adipate, a liquid polydimethyl siloxane, mineral oil, etc.); and

Optionally, at least one hydrophobic and/or hydrophilic silica (i.e. Sipernat D17, Aerosil R972, Aerosil R974, Aerosil 200, Sipernat 22S, Sipernat 50S, Syloid 244, Syloid 72, Syloid 63, etc.) Examples of such auxiliary substances are set forth in co-pending Applications for U.S. Letters Patent Publication Numbers 2004/0071742 and 2004/0072719 filed on Oct. 10, 2002 and published on Apr. 20, 2004.

Utility of the Spray-Dried Composition

As described herein, the present invention is well suited for use in a variety of all dry (anhydrous) products: powder laundry detergent, fabric softener dryer sheets, household cleaning dry wipes, powder dish detergent, floor cleaning cloths, or any dry form of personal care products (e.g. shampoo powder, deodorant powder, foot powder, soap powder, baby powder), etc. Because of high fragrance and/or active agent concentration in the spray-dried products of the present invention, characteristics of the aforementioned consumer dry products will not be adversely affected by a small dosage of the spray-dried products.

The spray drying inlet temperature is in the range of about 150° C. and about 240° C., preferably between about 170° C. and about 230° C., more preferably between about 190° C. and 220° C.

The present invention imparts a consumer benefit specifically relating to the different phase fragrance and/or benefit agent release: long-lasting benefit and/or fragrance perception via control release from capsules and relative immediate benefit and/or fragrance perception via release from water-soluble matrix dissolution. Also the change of sensory perception can be achieved due to the fragrance encapsulated in capsules could be different from that encapsulated in spray dry matrix. Finally, the high fragrance and/or benefit agent shelf life stability is made possible when spray-dried particles are placed in anhydrous bases with a minimal leaching.

These and additional modifications and improvements of the present invention may also be apparent to those with ordinary skill in the art. The particular combinations of elements described and illustrated herein are intended only to represent a certain embodiment of the present invention and are not intended to serve as limitations of alternative articles within the spirit and scope of the invention. All materials are reported in weight percent unless noted otherwise. As used herein all percentages are understood to be weight percent.

All U.S. Patents and patent applications cited herein are incorporated by reference as if set forth herein in their entirety.

### EXAMPLE A

The following fragrance composition was prepared:

| | C log₁₀P | Molecular | Parts by |
|---|---|---|---|
| Fragrance Component | value | Weight | Weight |
| ethyl undecylenate | 4.888 | 212.34 | 3.0 |
| geranyl anthranilate | 4.216 | 273.38 | 7.5 |
| α-irone | 3.820 | 206.33 | 6.3 |
| phenyl ethyl benzoate | 4.058 | 226.28 | 3.2 |
| d-limonene | 4.232 | 136.24 | 3.2 |
| cis-p-t-butylcyclohexyl acetate | 4.019 | 198.31 | 5.8 |
| amyl cinnamic aldehyde | 4.324 | 202.30 | 7.3 |
| hexyl cinnamic aldehyde | 5.473 | 216.33 | 12.6 |
| hexyl salicylate | 5.260 | 222.29 | 12.6 |

### EXAMPLE B

The following fragrance composition was prepared:

| | C log₁₀P | Molecular | Parts By |
|---|---|---|---|
| Fragrance Component | value | Weight | Weight |
| β-phenyl ethanol | 1.183 | 122.17 | 2.6 |
| benzyl acetate | 1.960 | 150.17 | 1.5 |
| α-irone | 3.820 | 206.33 | 6.3 |
| phenyl ethyl benzoate | 4.058 | 226.28 | 3.2 |
| d-limonene | 4.232 | 136.24 | 3.2 |
| cis-p-t-butylcyclohexyl acetate | 4.019 | 198.31 | 5.8 |
| amyl cinnamic aldehyde | 4.324 | 202.30 | 7.3 |
| hexyl cinnamic aldehyde | 5.473 | 216.33 | 12.6 |
| cis-jasmone | 2.712 | 164.25 | 14.3 |
| Geraniol | 2.649 | 154.26 | 3.8 |
| hexyl salicylate | 5.260 | 222.29 | 12.6 |

### EXAMPLE 1

### Preparation of Uncoated or Cationic Polymer-Coated Capsule Slurry That Contain Encapsulated Fragrance for Spray Drying

In this example, capsule slurry containing encapsulated fragrance oil was prepared by the following method 50 parts by weight of the fragrance oil of Example A was admixed with 50 parts by weight of NEOBEE-M5 solvent thereby forming a ‘fragrance/solvent composition’. In a homogenizer as illustrated in FIGS. 11-A and 11-B of U.S. Pat. No. 6,042,792, and in FIGS. 7A to 7D and 8A to 8E of U.S. patent application Ser. No. 10/823,033, fragrance/solvent composition-containing microcapsules was prepared by interfacial polymerization of a microcapsule wall encapsulating fragrance/solvent composition droplets. To make the capsule slurry, a copolymer of acrylamide and acrylic acid was first dispersed in water together with a methylated melamine-formaldehyde pre-condensate. These two components were allowed to react under acidic conditions. The fragrance/solvent composition was then added into the solution and droplets of the desired size were achieved by high shear homogenization. Curing of the polymeric layer around the fragrance/solvent composition droplets was achieved by increasing the temperature to 50-85° C. The resulting capsule slurry contained 55% water, and 45% filled microcapsules (35% core consisting of 50% fragrance of Example A, and 50% NEOBEE M-5 and 10% microcapsule wall). The slurry composed of capsules either in the presence or the absence of cationic deposition polymer was then spray-dried at 190° C. inlet air temperature using a Niro dryer (available from Niro Inc.), resulting in a free flowing microcapsule powder with a particle size in the range between 5 um and 400 um.

### EXAMPLE 2

### Preparation of Uncoated or Cationic Polymer-Coated Capsule Slurry That Contain Encapsulated Fragrance in the Absence of Formaldehyde Scavenger for Spray Drying

In this example, capsule slurry containing encapsulated fragrance oil was prepared as Example 1, except the addition of urea was removed during the capsule-making process. The slurry composed of capsules was then spray-dried at 190° C. inlet air temperature using a Niro dryer, resulting in a free flowing microcapsule powder with a similar physical characteristic and fragrance loading compared to that of Example 1.

### EXAMPLE 3

### Preparation of Uncoated or Cationic Polymer-Coated Capsule Slurry That Contain Encapsulated Fragrance and Catalyst for Spray Drying

In this example, capsule slurry containing encapsulated fragrance oil was prepared as Example 1, except magnesium chloride at the concentration of 0.15 weight % was added in slurry during the capsule-making process. The slurry composed of capsules either in the presence or the absence of cationic polymers was then spray-dried at 190° C. inlet air temperature using a Niro dryer, resulting in a free flowing microcapsule powder with a similar physical characteristic and fragrance loading compared to that of Example 1.

### EXAMPLE 4

### Preparation of Uncoated or Cationic Polymer-Coated Capsule Slurry That Contain Encapsulated Fragrance and Datalyst in the Absence of Formaldehyde Scavenger for Spray Drying

In this example, capsule slurry containing encapsulated fragrance oil was prepared as Example 1, except magnesium chloride at the concentration of 0.15 weight % in slurry was added and urea addition was removed during the capsule-making process. The slurry composed of capsules either in the presence or the absence of cationic polymers was then spray-dried at 190° C. inlet air temperature using a Niro dryer, resulting in a free flowing microcapsule powder with a similar physical characteristic and fragrance loading compared to that of Example 1.

### EXAMPLE 5

### Preparation of Uncoated or Cationic Polymer-Coated Capsule Slurry That Contain Encapsulated Fragrance and Water-Soluble Polymer for Spray Drying

In this example, capsule slurry containing encapsulated fragrance oil was prepared as described in either Example 1, or Example 2, or Example 3, or Example 4, except Maltrin M100 was added at 15 weight % in the slurry prior to drying. The slurry composed of capsules either in the presence or the absence of cationic polymers was then spray-dried at 190° C. inlet air temperature using a Niro dryer, resulting in a free flowing microcapsule powder with a similar physical characteristic but lower fragrance loading compared to that of Example 1 due to the introduced solids from Maltrin M100.

### EXAMPLE 6

Preparation of Uncoated or Cationic Polymer-Coated Capsule Slurry That Contain Encapsulated Fragrance and Non-Confined Fragrance Along with Non-Emulsifying Water-Soluble Polymer for Spray Drying

In this example, capsule slurry containing encapsulated and non-confined fragrance oil was prepared according to the method described in U.S. patent application Ser. No. 10/823,033, where TWEEN 20 was used as an oil-in-water type emulsifier. The weight ratio of encapsulated fragrance oil of Example A and non-confined fragrance oil of Example B was not limited to an equal 1 to 1 part. Maltrin M100 was added by low-shear mixing into the slurry at the same weight % as the non-confined fragrance oil prior to the drying. The slurry composed of capsules either in the presence or the absence of cationic polymers was then spray-dried at 190° C. inlet air temperature using a Niro dryer, resulting in a free flowing microcapsule powder with a similar physical characteristic but lower fragrance loading compared to that of Example 1 due to the introduced solids from Maltrin M100

### EXAMPLE 7

### Preparation of Uncoated or Cationic Polymer-Coated Capsule Slurry That Contain Encapsulated Fragrance and Non-Confined Fragrance Emulsified by Self-Emulsifying Water-Soluble Polymer for Spray Drying

In this example, capsule slurry containing encapsulated fragrance oil was prepared as described in either Example 1, or Example 2, or Example 3, or Example 4. A separate oil-in-water emulsion was prepared by emulsifying non-confined fragrance oil of Example B in a solution containing 30 weight % of N-LOK via high shearing mixing. The oil-in-water fragrance emulsion was then added into capsule slurry with a low-shear mixing prior to drying. The slurry composed of capsules either in the presence or the absence of cationic polymers was then spray-dried at 190° C. inlet air temperature using a Niro dryer, resulting in a free flowing microcapsule powder with a similar physical characteristic but lower fragrance loading compared to that of Example 1 due to the result of introduced solids from N-LOK.

### EXAMPLE 8

### Effect of Neat Fragrance, Capsule Slurry, and Spray Dry Capsules in the Dryer Sheet Fabric Conditioning Coating Mix

A model dryer sheet fabric conditioning coating mix consisting of a mixture of quaternary ammonium compounds and fatty materials was melted at approximately 82° C. prior to the incorporation of neat fragrance at 5 weight %, capsule slurry at 5 weight % fragrance equivalency, and spray dry capsules in referred in the Example 1 at 5 or 6 weight % fragrance equivalency. Initial set time and final set time were determined by monitoring the onset of molten softening coating mix from its liquid to solid state. Set temperature was the temperature determined by a Differential Scanning Calorimeter for the molten coating mix changing from amorphous state to crystalline state.

As can be seen from the table below, the addition of neat fragrance at 5 weight % in the softening coating mix resulted in an undesired increase of both initial and final set time compared to those of the un-fragranced coating mix. The addition of capsule slurry at 5 weight % fragrance equivalency resulted in even higher increase of both initial and final set time than those of the coating mix containing neat fragrance alone. In either case whether adding 5 weight % neat fragrance or capsule slurry at 5 weight % neat equivalence is unacceptable for manufacturing processing due to the significant alteration of the physiochemical property of the softening coating mix. Whereas the addition of spray-dried capsules at either 5 weight % or 6 weight % fragrance equivalence into the softening coating mix only resulted in relatively smaller changes on initial set time and final set time as well as set temperature. The physiochemical property of the softening coating mix that contained spray-dried capsules at 6 weight % fragrance equivalence appeared to be more similar to that of the unfragrance coating mix than any other variants.

| Fragrance presentation | Initial | Final | Set |
|---|---|---|---|
| and concentration in | set time | set time | temperature |
| softening coating mix | (second) | (second) | (° C.) |
| Un-fragrance softening coating mix | 23 | 96 | 63 |
| Coating mix containing 5% neat | 55 | >120 | N/A |
| fragrance | | | |
| Coating mix containing capsule | 90 | >600 | N/A |
| slurry at 5 weight % fragrance | | | |
| equivalence | | | |
| Coating mix containing spray-dried | 21 | 74 | 67 |
| capsules at 5 weight % fragrance | | | |
| equivalence | | | |
| Coating mix containing spray-dried | 20 | 81 | 65 |
| capsules at 6 weight % fragrance | | | |
| equivalence | | | |

### EXAMPLE 9

### Sensory Performance on Drying Fabric Treated with Tumble Dryer Sheets That Contain Spray-Dried Capsules

Twenty-two hand towels (approximately 2200 gm total) were machine tumble dried with a 6.4″ by 9″ dyer sheet, immediately after machine wash and rinsing. Three different tumble dryer sheet variants were made as (A) control where the fabric softening coating mix contained 7 weight % neat fragrance, (B) variant where the fabric softening coating mix contained 5 weight % fragrance equivalent spray-dried capsules of Example 5, (C) variant where the fabric softening coating mix contain 6 weight % fragrance equivalent (5 weight % encapsulated and 1 weight % non-confined) spray-dried capsules of Example 6. Dry towels were evaluated by a panel of ten people using the Label Magnitude Scale (LMS) from 0 to 99, where 3=barely detectable, 7=weak, 16=moderate, and 32=strong. Sensory scores were recorded before and after towels in plastic bags were rubbed by hand.

As can be seen from the table below, the (B) variant containing spray-dried capsules showed directionally higher pre-rub intensity rating and significantly higher post-rub intensity rating than those of the (A) control sample, even only at 71.4% fragrance equivalency. Comparing both pre-rub and post-rub intensity ratings of the (C) variant to the (B) variant, data suggested that the additional 1% non-confined fragrance in the spray dry matrix resulted in a directional better sensory performance.

| Fragrance presentation and | | |
|---|---|---|
| concentration in the fabric | Pre-rub sensory | Post-rub sensory |
| conditioning coating base | intensity rating | intensity rating |
| A control (7% neat fragrance) | 5.3 | 3.4 |
| B variant (5% capsule encapsulated | 7.6 | 11.6 |
| fragrance via spray dry) | | |
| C variant (5% capsule encapsulated | 9.3 | 12.2 |
| and 1% non-confined fragrance via | | |
| spray dry) | | |

We claim:

1. A composition in spray-dried form comprising:
a plurality of microcapsules each of which is composed of a polymeric rupturable external wall enclosing an active material dispersed in a spray-dry carrier wherein the carrier comprises a water-soluble material with non-emulsifying and/or self-emulsifying properties, an optional functional additive and an optional non-confined active material with the proviso that when a non-emulsifying water-soluble material is employed in combination with a non-confined active material an emulsifier must be used in the combination; and wherein the composition of each of the cores of each of said microcapsules being (A) the same and/or different from one another and (B) the same and/or different from the non-confined active material optionally present in said spray-dry carrier wherein the weight % of active material initially contained in each of the microcapsules is from about 5% to 98% by weight of the microcapsules.

2. The spray-dried composition of claim 1 wherein the self-emulsifying water-soluble material is selected from the group consisting of modified starches, proteins, gum arabic and combinations thereof.

3. The spray-dried composition of claim 1 wherein the non-emulsifying water-soluble material is selected from the group consisting of carbohydrates including mono, di and trisaccharides, gums, cellulose derivatives, maltodextrins, corn syrup solids, glycols, synthetic polymers and/or copolymers and combinations thereof.

4. The composition of claim 1 wherein the optional functional additive is a capsule deposition aid selected from the group consisting of cationic starches such as Hi-CAT CWS42, cationic guars such as Jaguar C-162, cationic amino resins, cationic urea resins, hydrophobic quaternary amines and combinations thereof.

5. The composition of claim 4 wherein the capsule deposition aid is present in the amount from about 0.01% to about 25%.

6. The composition of claim 1 wherein the optional functional additive is a humectant selected from the group consisting of polyhydric alcohols such a glycerin, propylene glycol, maltitol, alkoxylated nonionic polymers such as polyethylene glycols, polypropylene glycols.

7. The composition of claim 6 wherein the humectant is present in the amount of about 0.01% to about 25%.

8. The composition of claim 1 wherein the optional functional additive is a crosslinker selected from the group consisting of aminoplasts such as melamine-formaldehyde, urea formaldehyde, aldehydes such as acetaldehyde, dialdehydes such as glutaraldehyde, poly-substituted carboxylic acids and derivatives such as acid chlorides, anyhydrides, isocyanates, tannin/tannic acid inorganic crosslinkers such as Ca²⁺.

9. The composition of claim 8 wherein the crosslinker is present in the amount of about 0.01% to about 10%.

10. The composition of claim 1 wherein the optional functional additive is a viscosity control agent selected from the group consisting of modified cellulose polymers such as methylcellulose, hydoxyethylcellulose, cross-linked acrylate polymers and hydrophobically modified polyethers.

11. The composition of claim 10 wherein the viscosity control agent is present in the amount of about 0.01% to about 25%.

12. The composition of claim 1 wherein the optional functional additive is a free flow agent of hydrophobic and/or hydrophilic silica.

13. The composition of claim 12 wherein the free flow agent is present in the amount of about 0.01% to about 10%.

14. The spray-dried composition of claim 1 wherein the emulsifier is at least one emulsifier selected from the group consisting of non-ionic emulsifers, anionic emulsifiers and zwitterionic emulsifiers, each of which has an HLB value of from about 6 to about 40 with the provisos that:
(a) when using a non-ionic emulsifier, the HLB value is in the range of from about 6 to about 20;
(b) when using an anionic emulsifier, the HLB value is in the range of from about 10 to about 40; and
(c) when using a zwitterionic emulsifier, the HLB value is in the range of from about 6 to about 12.

15. The spray-dried composition of claim 14 wherein the emulsifier is a non-ionic emulsifier having a HLB value in the range of from about 6 to about 20.

16. The spray-dried composition of claim 15 wherein the non-ionic emulsifier is polyoxyethylene (20) sorbitan monolaurate.

17. The spray-dried composition of claim 14 wherein the emulsifier is a zwitterionic emulsifier having a HLB value in the range of from about 6 to about 12.

18. The spray-dried composition of claim 17 wherein the zwitterionic emulsifier is a phosphatidylcholine.

19. The spray-dried composition of claim 14 wherein the emulsifier is an anionic emulsifier having a HLB value in the range of from about 10 to about 40.

20. The spray-dried composition of claim 19 wherein the anionic emulsifier is the sodium salt of n-dodecyl sulfate.

21. The composition of claim 1 wherein the active material is selected from the group consisting of fragrances, flavoring agents, fungicide, brighteners, antistatic agents, wrinkle control agents, fabric softener actives, hard surface cleaning actives, skin and/or hair conditioning agents, malodour counteractant, antimicrobial actives, UV protection agents, insect repellents, animal/vermin repellants, flame retardants, and mixtures thereof.

22. The active material of claim 21 wherein the active material is not water soluble.

23. The composition of claim 21 wherein the active material is from about 10% to about 98% of the composition.

24. The composition of claim 21 wherein the active material contained in each of the microcapsules has a Clog P value of preferable greater than 3.3 at greater than 60% by weight, more preferably has a Clog P value greater than 4.0 at greater than 80% by weight, and most preferably has a Clog P value greater than 4.0 at greater than 90% by weight.

25. The composition of claim 1 wherein the polymeric rupturable exterior wall comprises crosslinked or non-crosslinked polymers selected from the group consisting of vinyl, acrylic and polysaccharide polymers and proteins with formaldehyde, epoxy, acid anhydrides, acid chlorides, esters, carboxylic acid, amine, alcohol, thial functional groups.

26. The composition of claim 1 wherein the microcapsules are further coatedby a cationic polymer.

27. The composition of claim 26 wherein the cationic polymer is selected from the group consisting of polysaccharides, cationically modified starch and cationically modified guar, polysiloxanes, poly diallyl dimethyl ammonium halides, copolymers of poly diallyl dimethyl ammonium chloride and vinyl pyrrolidone, acrylamides, imidazoles, imidazolinium halides and imidazolium halides and poly vinyl amine and its copolymers with N-vinyl formamide.

28. The composition of claim 1 which is incorporated into a product selected from the group consisting of a personal care, fabric care, household care and cleaning products.

29. The composition of claim 28 wherein the personal care product is selected from the group consisting of bar soaps, shampoo powder, deodorant powder, foot powder, soap powder, baby powder, etc.

30. A method of imparting an effective amount of active material into a spray-dried product comprising the steps of:
providing an active material;
encapsulating the active material with a polymer optionally in the presence of a catalyst and in the absence of urea to form a polymer encapsulated active material;
providing an aqueous solution comprising water and a spray-dry carrier matrix comprising a water-soluble material with non-emulsifying and/or self-emulsifying properties, an optional functional additive and an optional non-confined active material with the proviso that when a non-emulsifying water-soluble material is employed in combination with a non-confined active material an emulsifier must be used in the combination;
admixing the plurality of microcapsules with the aqueous solution; and
spray drying the solution with a spray drying inlet temperature is from about 150° C. to about 240° C. to form the spray-dried product.

31. The method of claim 30 wherein the self-emulsifying water-soluble material is selected from the group consisting of modified starches, proteins, gum arabic and combinations thereof.

32. The method of claim 30 wherein the non-emulsifying water-soluble material is selected from the group consisting of carbohydrates including mono, di and trisaccharides, gums, cellulose derivatives, maltodextrins, corn syrup solids, glycols, synthetic polymers and/or copolymers and combinations thereof.

33. The method of claim 30 wherein the optional functional additive is a capsule deposition aid selected from the group consisting of cationic starches such as Hi-CAT CWS42, cationic guars such as Jaguar C-162, cationic amino resins, cationic urea resins, hydrophobic quaternary amines and combinations thereof.

34. The method of claim 30 wherein the optional functional additive is a humectant selected from the group consisting of polyhydric alcohols such a glycerin, propylene glycol, maltitol, alkoxylated nonionic polymers such as polyethylene glycols, polypropylene glycols.

35. The method of claim 30 wherein the optional functional additive is a crosslinker selected from the group consisting of aminoplasts such as melamine-formaldehyde, urea formaldehyde, aldehydes such as acetaldehyde, dialdehydes such as glutaraldehyde, poly-substituted carboxylic acids and derivatives such as acid chlorides, anyhydrides, isocyanates, tannin/tannic acid inorganic crosslinkers such as Ca²⁺.

36. The method of claim 30 wherein the optional functional additive is a viscosity control agent selected from the group consisting of modified cellulose polymers such as methylcellulose, hydoxyethylcellulose, cross-linked acrylate polymers and hydrophobically modified polyethers.

37. The method of claim 30 wherein the optional functional additive is a free flow agent of hydrophobic and/or hydrophilic silica.

38. The method of claim 37 wherein the hydrophobic silica may be dispersed in the non-confined material and hydrophilic silica may be dispersed in water prior to spray drying and/or both hydrophobic and hydrophilic silicas may be added into finished spray-dried powders.

39. The method of claim 30 wherein the emulsifier is at least one emulsifier selected from the group consisting of non-ionic emulsifers, anionic emulsifiers and zwitterionic emulsifiers, each of which has an HLB value of from about 6 to about 40 with the provisos that:
(d) when using a non-ionic emulsifier, the HLB value is in the range of from about 6 to about 20;
(e) when using an anionic emulsifier, the HLB value is in the range of from about 10 to about 40; and
(f) when using a zwitterionic emulsifier, the HLB value is in the range of from about 6 to about 12.

40. The method of claim 39 wherein the emulsifier is a non-ionic emulsifier having a HLB value in the range of from about 6 to about 20.

41. The method of claim 40 wherein the non-ionic emulsifier is polyoxyethylene (20) sorbitan monolaurate.

42. The method of claim 39 wherein the emulsifier is a zwitterionic emulsifier having a HLB value in the range of from about 6 to about 12.

43. The method of claim 42 wherein the zwitterionic emulsifier is a phosphatidylcholine.

44. The method of claim 39 wherein the emulsifier is an anionic emulsifier having a HLB value in the range of from about 10 to about 40.

45. The method of claim 44 wherein the anionic emulsifier is the sodium salt of n-dodecyl sulfate.

46. The method of claim 39 wherein the microcapsules are encapsulated by a polymer selected from the group consisting of vinyl polymer; an acrylate polymer, an amine-containing and/or generating polymer with primary and/or secondary amine groups, melamine-formaldehyde; urea formaldehyde and mixtures thereof.

47. The method of claim 46 wherein the microcapsules are further coated with a cationic polymer.

48. The method of claim 47 wherein the cationic polymer is selected from polysaccharides, cationically modified starch and cationically modified guar, polysiloxanes, poly diallyl dimethyl ammonium halides, copolymers of poly diallyl dimethyl ammonium chloride and vinyl pyrrolidone, acrylamides, imidazoles, imidazolinium halides and imidazolium halides.

49. The method of claim 48 wherein the optional catalyst is selected from the group consisting of mineral acids, ammonium salts, oxalic and hydroxyl-substituted acids, glycolic tartaric and citric acid, amine hydrochlorides, metal salts, zinc nitrate, zinc chloride, magnesium chloride and combinations thereof.
